(19) United States
(12) Patent Application Publication
(10) Pub. No.: US 2014/0031676 A1
(43) Pub. Date: Jan. 30, 2014

(54) EMBEDDED 3D MODELLING

(71) Applicants: Olivier Pierre Nempont, Suresnes (FR); Raoul Florent, Ville D'Avray (FR); Pascal Yves Francois Cathier, Asnieres-Sur-Seine (FR)

(72) Inventors: Olivier Pierre Nempont, Suresnes (FR); Raoul Florent, Ville D'Avray (FR); Pascal Yves Francois Cathier, Asnieres-Sur-Seine (FR)

(73) Assignee: KONINKLIJKE PHILIPS N.V., EINDHOVEN (NL)

(21) Appl. No.: 14/110,970

(22) PCT Filed: Apr. 5, 2012
(86) PCT No.: PCT/IB2012/051700

§ 371 Date: Oct. 10, 2013

(30) Foreign Application Priority Data
Apr. 12, 2011 (EP) 11305428.2

(51) Int. Cl. A61B 6/12

(52) U.S. Cl. CPC A61B 6/12
USPC 600/424

(57) ABSTRACT

The present invention relates to an image processing device for guidance support, a medical imaging system for providing guidance support, a method for guidance support, a method for operating an image processing device for guidance support, as well as a computer program element, and a computer readable medium. In order to provide enhanced and easily perceptible information about the actual situation, it is proposed to provide (110) 3D data (112) of a region of interest of an object, to provide (114) image data (116) of at least a part of the region of interest, wherein a device is located at least partly within the region of interest, to generate (118) a 3D model (120) of the device from the image data, and to provide (122) data for a model-updated 3D image (124) by embedding (126) the 3D model within the 3D data.

### FIELD OF THE INVENTION

The present invention relates to an image processing device for guidance support, a medical imaging system for providing guidance support, a method for guidance support, a method for operating an image processing device for guidance support, as well as a computer program element, and a computer readable medium.

### BACKGROUND OF THE INVENTION

Guidance support can be provided, for example, to a surgeon during an interventional procedure, such as an examination or operation of a patient. One example for an interventional procedure is the placing of a stent in a so-called minimal invasive procedure. In order to provide the surgeon with information about the current situation, which is, needless to say, generally not directly visible for the surgeon himself, image data of a region of interest of an object, for example of a region of a patient, is provided on a display. For example, US 2010/0061603 A1 describes the acquisition of 2D live images, which are combined with pre-operationally 3D image data and displayed as image composition to a user. It has been shown that the information generated prior to the operation may deviate from the current situation. It has been shown further that for providing improved image information about the current situation, the user has to rely on the acquired image data.

### SUMMARY OF THE INVENTION

It is an object of the present invention to provide enhanced and easily perceptible information about the actual situation.

The object of the present invention is solved by the subject-matter of the independent claims, wherein further embodiments are incorporated in the dependent claims.

It should be noted that the following described aspects of the invention apply also for the image processing device, the medical imaging system, the method for guidance support, the method for operating an image processing device for guidance support, the computer program element, and the computer readable medium.

According to an aspect of the invention, an image processing device for guidance support is provided, comprising a processing unit, an input unit, and an output unit. The input unit is adapted to provide 3D data of a region of interest of an object, and to provide image data of at least a part of the region of interest, wherein a device is arranged at least partly within the region of interest. The processing unit comprises a generation unit to generate a 3D model of the device from the image data. The processing unit comprises an embedding unit to embed the 3D model within the 3D data. The output unit is adapted to provide a model-updated 3D image with the embedded 3D model.

According to the present invention, the term “guidance support” refers to providing information to a user, for example a surgeon or an interventional radiologist which supports, helps or facilitates any intervention where a device or other equipment or part has to be moved or steered inside a volume while it Is not directly visible to the user. The “guidance support” can be any type of information providing a better understanding about the current situation, preferably by visible information.

According to an exemplary embodiment, the image data comprises at least one 2D image and the generation unit is adapted to generate the 3D model from the at least one 2D image. The generation unit is adapted to generate a 3D representation of the region of interest from the 3D data, and the processing unit is adapted to embed the 3D model within the 3D representation.

According to a further aspect of the invention, a medical imaging system for providing guidance support is provided, comprising an image acquisition arrangement, a display unit, and an image processing device according to the above mentioned aspect and exemplary embodiment. The image acquisition arrangement is adapted to acquire the image data and to provide the data to the processing unit. The output unit is adapted to provide the model-updated 3D image to the display unit, and the display unit is adapted to display the model-updated 3D image.

According to an exemplary embodiment, the image acquisition arrangement is an X-ray imaging arrangement with an X-ray source and an X-ray detector. The X-ray imaging arrangement is adapted to provide 2D X-ray images as image data.

According to a further aspect of the invention, a method for guidance support is provided, comprising the following steps:

a) providing 3D data of a region of interest of an object;

b) providing image data of at least a part of the region of interest, wherein a device is located at least partly within the region of interest;

c) generating a 3D model of the device from the image data; and

d) providing data for a model-updated 3D image by embedding the 3D model within the 3D data.

According to an exemplary embodiment of the invention, a spatial relationship between the 3D model and the 3D data is predetermined, and for the embedding, the 3D model is adjusted accordingly.

According to a further exemplary embodiment of the invention, predetermined features of the device and/or the object are detected in the model-updated 3D image.

For example, the predetermined features are highlighted in the model-updated 3D image.

For example, measurement data of the detected features in relation to the object is determined and the measurement data is provided to define and/or adapt a steering or guiding strategy of an intervention.

According to a further aspect of the invention, a method for operating an image processing device for guidance support is provided, comprising the following steps:

providing 3D data of a region of interest of an object from an input unit to a processing unit;

providing image data of at least a part of the region of interest from the input unit to the processing unit, wherein a device is arranged at least partly within the region of interest;

generating a 3D model of the device from the image data by the processing unit; and

embedding the 3D model within the 3D data by the processing unit to provide a model-updated 3D image via an output unit.

It can be seen as the an aspect of the invention to take the image data, for example image data reflecting the current situation, such as live fluoroscopy images, as a basis for modeling the device itself. Thus, a model of the device is generated which is in exact congruence with the current situation, i.e. which represents the current situation. This so-to-speak live model is then shown in the context of 3D data in order to provide the user with easily perceptible and precise information about the current situation.

These and other aspects of the present invention will become apparent from and elucidated with reference to the embodiments described hereinafter.

### BRIEF DESCRIPTION OF THE DRAWINGS

Exemplary embodiments of the invention of the invention will be described in the following with reference to the following drawings.

FIG. 1 illustrates an image processing device according to an exemplary embodiment of the invention.

FIG. 2 illustrates a further example of an image processing device according to the invention.

FIG. 3 illustrates a medical imaging system according to an exemplary embodiment of the invention.

FIG. 4 illustrates a method for guidance support according to an exemplary embodiment of the invention.

FIGS. 5 to 10 show further examples of exemplary embodiments of a method according to the invention.

FIG. 11 illustrates a method for operating an image processing device according to an exemplary embodiment of the invention.

FIGS. 12 to 15 show further aspects of an embodiment according to the invention.

FIG. 16 shows a further example of a method according to the invention.

### DETAILED DESCRIPTION OF EMBODIMENTS

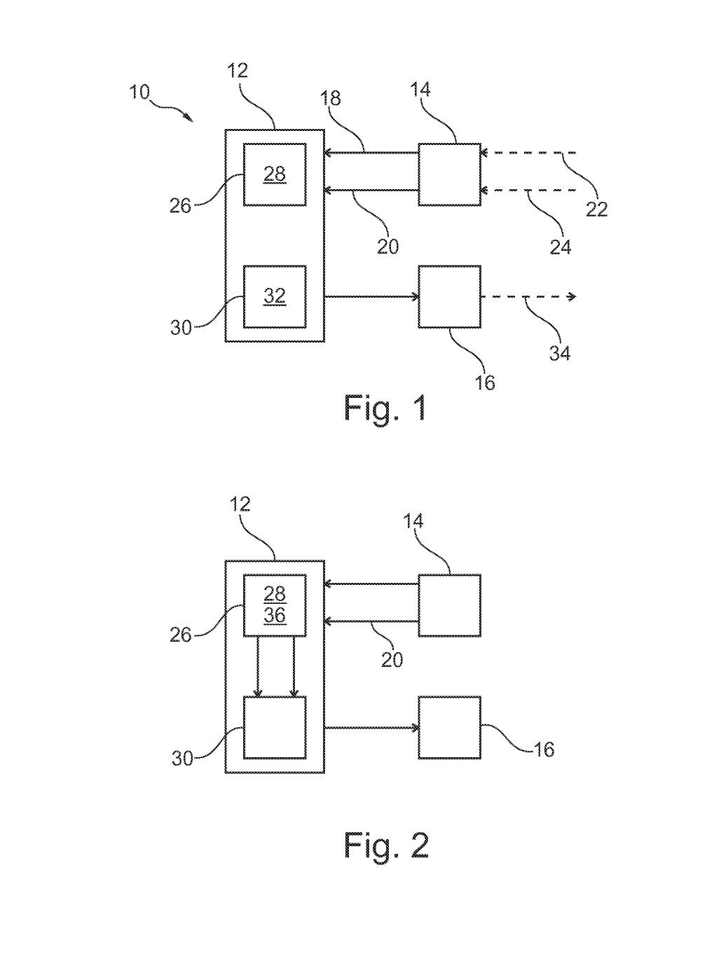

FIG. 1 illustrates an image processing device 10 for guidance support with a processing unit 12, and input unit 14, and an output unit 16. The input unit 14 is adapted to provide 3D data of a region of interest of an object. The provision of the 3D data is indicated with a first arrow 18. The input unit 14 is further adapted to provide image data of at least a part of the region of interest. The provision of the image data is indicated with a second arrow 20. In the image data, a device is arranged at least partly within the region of interest.

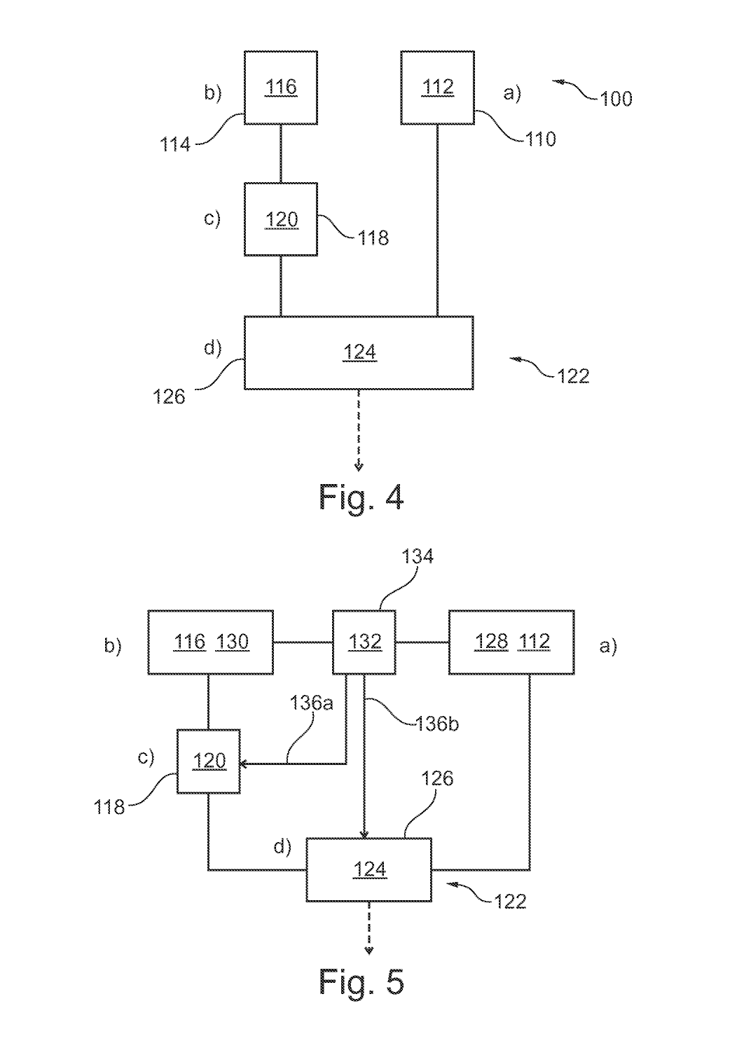

For example, the 3D data 18 and the image data 20 can be provided to the input unit 14 from external sources, as indicated with respective dotted arrows 22 and 24. For example, the 3D data 18 can be provided from a storage unit, not further shown; the image data 20 can be provided from an image acquisition device, as will be explained with reference to FIG. 3 as an example.

The processing unit 12 comprises a generation unit 26 to generate a 3D model 28 of the device from the image data 20. The processing unit 12 further comprises an embedding unit 30 to embed the 3D model 28 within the 3D data 18. Thus, data for a model-updated 3D image 32 with the embedded 3D model is achieved. The output unit 16 is adapted to provide the model-updated 3D image 32, for example to a further external component, as indicated with dotted arrow 34.

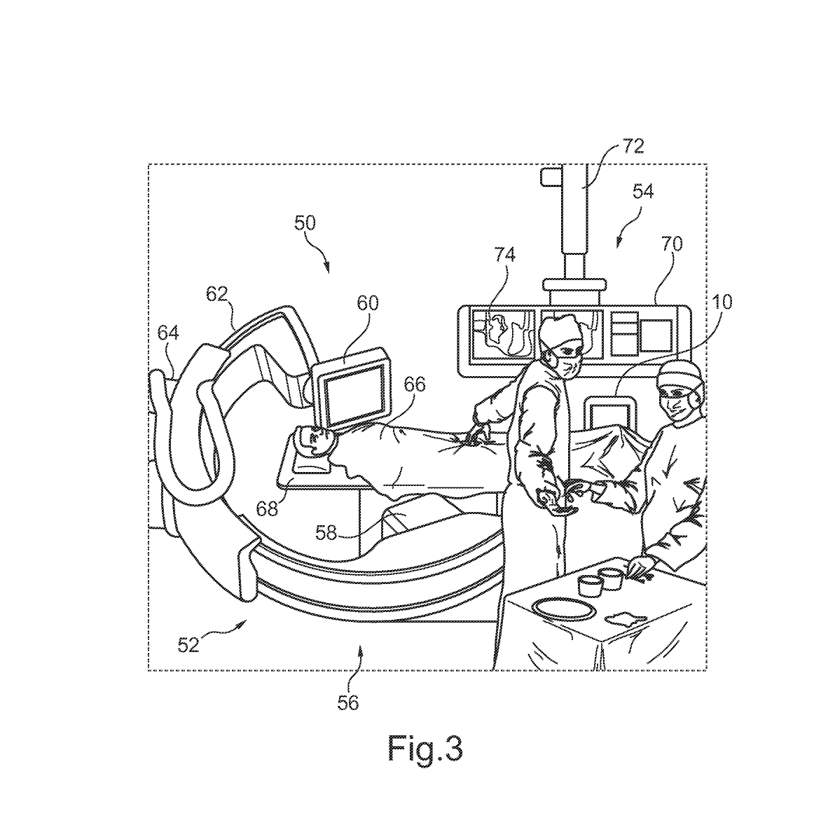

According to a further exemplary embodiment, shown in FIG. 2, the image data 20 comprises at least one 2D image. The generation unit 26 is adapted to generate a 3D representation 36 of the region of interest from the 3D data. The embedding unit 30 is adapted to embed the 3D model 28 within the 3D representation 36. It must be noted that the similar features are indicated with same reference numerals in FIG. 2 compared with FIG. 1.

FIG. 3 shows an example for a medical imaging system 50 for providing guidance support, comprising an image acquisition arrangement 52, and image processing device 10 according to the above described exemplary embodiments, and a display unit 54. The image acquisition arrangement 52 is adapted to acquire the image data, for example the image data 20 of FIGS. 1 and 2, and to provide the data to the processing unit, for example the processing unit 12. The output unit (not further shown) of the image processing device 10 is adapted to provide the model-updated 3D image to the display unit 54. The display unit 54 is adapted to display the model-updated 3D image.

FIG. 3 shows an X-ray imaging arrangement 56 as the image acquisition arrangement 52. The X-ray imaging arrangement 56 comprises an X-ray source 58, and an X-ray detector 60. The X-ray imaging arrangement 56 is adapted to provide 2D X-ray images as image data 20, for example. The X-ray imaging arrangement 56 is shown as a C-arm structure with the X-ray source 58 and the X-ray detector 60 on opposing ends of the C-arm structure 62. The C-arm structure 62 is mounted via a support structure 64, which allows a rotational movement of the C-arm as well as a sliding movement of the C-arm structure 62 in the support 64. The support 64 is further supported by a support base, for example with a suspending base, mounted to a ceiling of an operational room, for example. The C-arm is mounted such that different acquisition directions are possible in order to acquire image information about an object, for example a patient 66 from different directions. Further, a support in form of a table 68 is provided to support the patient, for example in a horizontal manner. Thus, the table 68 can serve as an operational table or a table during an examination procedure.

The display unit 54 is shown with several display areas, which can be arranged as different monitors or also with different sub-areas of a larger monitor. The different sub-areas form a display area 70. The display unit 54 can be suspended from a ceiling via a display support structure 72, for example.

It must be noted that the X-ray imaging arrangement 56 is shown in form of a C-arm device as an example only. Of course, other imaging modalities can be provided, for example other movable arrangements, such as a CT with a gantry, or static imaging devices, for example those where the patient is arranged in a horizontal manner as well as those where the patient is in an upright standing position, such as mammography imaging devices.

According to a further example, although not shown, the image acquisition arrangement is provided as an ultrasonic image acquisition arrangement to provide ultrasonic images instead of X-ray images for the image data 20.

The medical imaging system 50 of FIG. 3 will also be explained in its functionality with reference to the following drawings in which exemplary embodiments of a method to be performed by the medical imaging system and/or the image processing device 10 with reference to the following drawings. As indicated in FIG. 3, the medical imaging system 50 is adapted to display enhanced information about the current situation in form of a displayed image 74, for example, showing the model-updated 3D image 32.

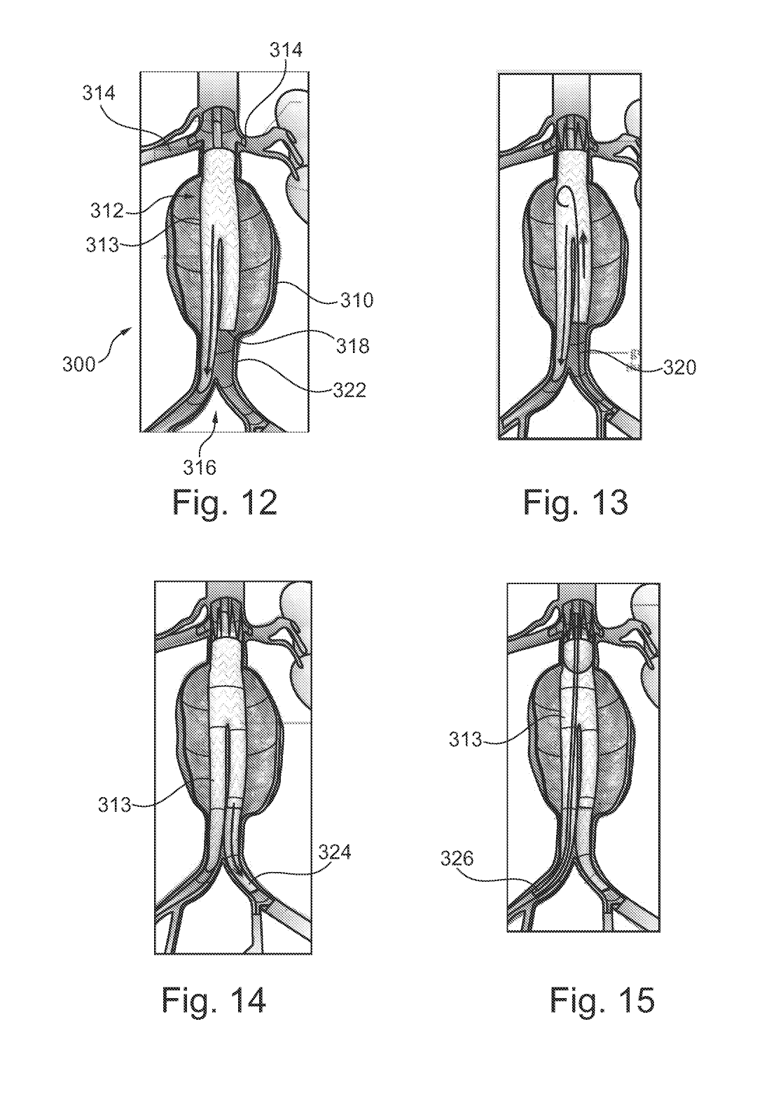

The medical imaging system 50 and the method described in the following can be used, for example, during endovascular surgery procedures, such as endovascular aneurism repair, which will be explained further below with reference to FIG. 12 et seq.

When defining that the image data, for example live 2D image data, is having a device arranged at least partly within the region of interest, the device can be a stent, a catheter, or a guide-wire, for example, or any other interventional tool or endo-prosthesis. It is not necessary to fully arrange the device in the region of interest, but only a part of it as a minimum. This part has to be sufficient in order to be able to generate a model therefrom in three dimensions.

For example, the model of the device can be static. According to another example, it's a dynamic model. Of course, it is also possible to have a part of the model static and a part of the model dynamic, for example in case a part of the model relates to a (moving) guide-wire as a dynamic part and another part relates to an implant or prosthesis as a static part. However, it must be noted that moving relates primarily to the movement in relation to the object, but of course movement of the body or body parts, for example caused by breathing or heart beat related movements, can also be considered.

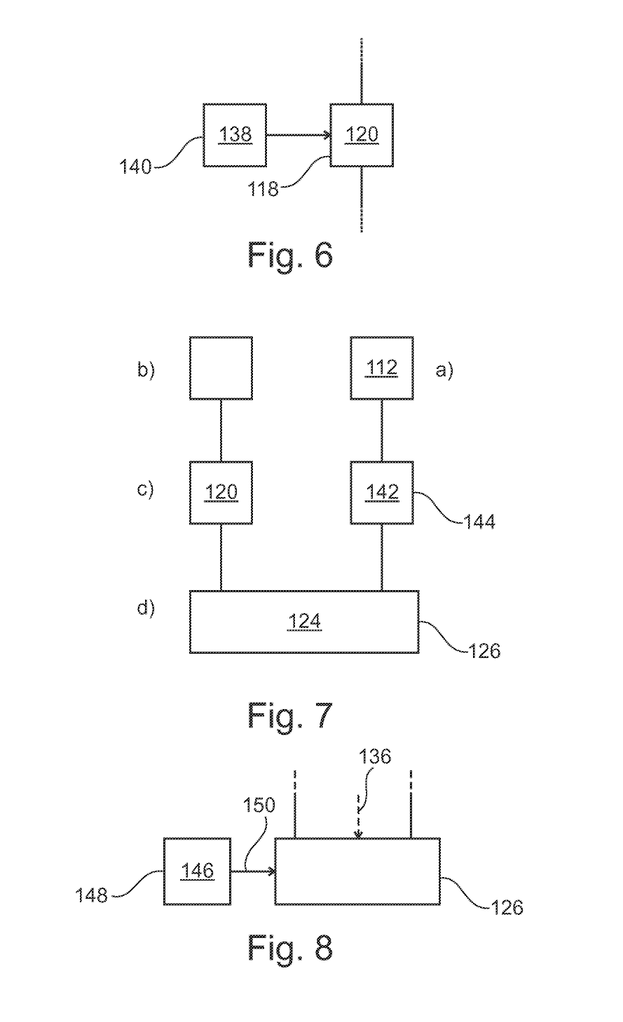

FIG. 4 shows a method 100 for guidance support, comprising the following steps. A first provision step 110 is provided in which 3D data 112 of a region of interest of an object is provided. In a second provision step 114, image data 116 of at least a part of the region of interest is provided, wherein a device is located at least partly within the region of interest. In a generation step 118, a 3D model 120 of the device is generated from the image data. In a third provision step 122, data for a model-updated 3D image 124 is provided by embedding 126 the 3D model within the 3D data 112.

The first provision step 110 is also referred to as step a), the second provision step 114 as step b), the generation step 118 as step c), and the third provision step 122 as step d).

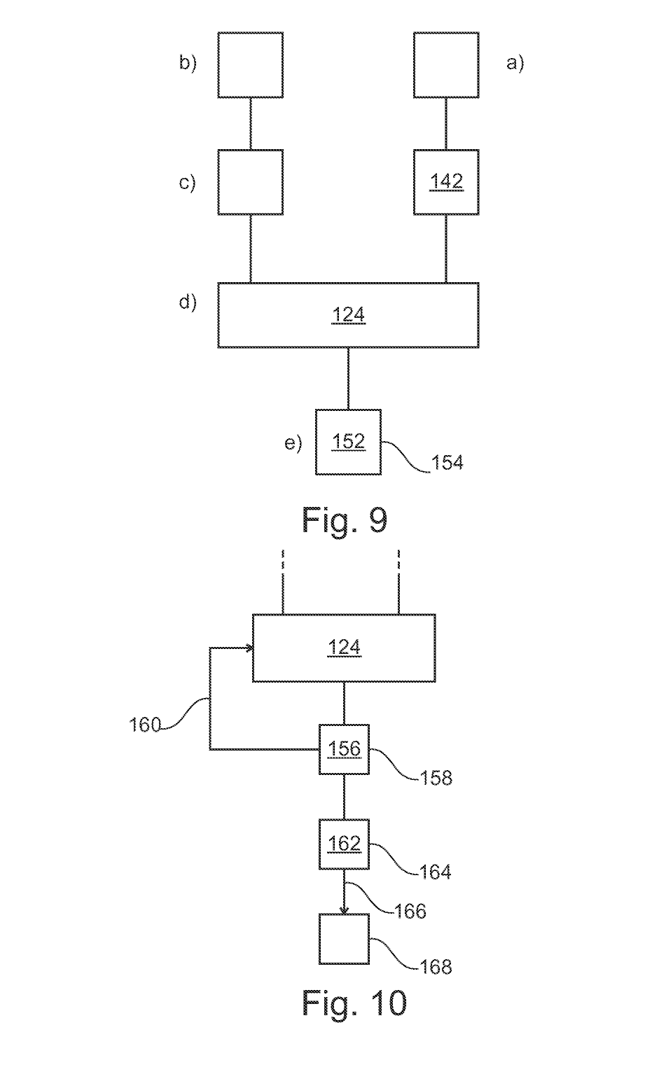

According to an exemplary embodiment, shown in FIG. 5, the 3D data 112 in step a) comprises a first frame of reference 128 and the image data 116 in step b) comprises a second frame of reference 130. For the embedding 126 in step d), a transformation 132 between the first frame of reference 128 and the second frame of reference 130 is determined in a determination sub-step 134. The transformation 132 is then applied to the 3D model 120. This application can be achieved, for example by applying the geometrical transformation 132 directly in step c), as indicated with first application arrow 136a or by applying the transformation to the embedding 126 in step d), as indicated with second application arrow 136b. This leads to a model-upgraded 3D image represented in the frame of reference 128. Of course the geometrical transform (or its inverse) can instead be applied to the 3D data 112, leading to a model-upgraded 3D image represented in the frame of reference 130. In fact it does not matter in which frame of reference the result 124 is represented, provided the frames of reference 130 and 128 are correctly aligned after geometrical transform 132. For that matter, geometrical transform 132 might even be split into two transforms, one to be applied onto frame of reference 128 and one to be applied onto frame of reference 130, providing the transform pair is such that after this dual transformation, the two frames of reference 128 and 130 spatially coincide.

For example, the 3D data 112 is registered with the image data 116.

According to a further example, the 3D data 112 in step a) is also referred to as first image data, and the image data 116 in step b) is referred to as second image data.

For example, the image data 116 comprises at least one 2D image.

According to a further example, shown in FIG. 6, for the modeling in step c), i.e. for the generating 118 of the 3D model 120, shape assumptions 138 are provided in a provision sub-step 140 to facilitate the modeling. For example, in correlation with the particular examination or interventional procedure, it can be expected that the object, i.e. the patient, shows certain shapes for certain anatomical structures, such as a vessel tree with certain shape forming depending from the respective location of the region of interest.

According to a further example, not further shown, the image data 20, or the so-to-speak second image data, comprises a set of live 2D images. Following, step c) comprises building or generating the 3D model from the set of live 2D images.

The model-updated 3D image 124 can be used as a steering guidance image.

Also with reference to FIG. 5, it is noted that the registration step of the first and second image data, i.e. the determination of the spatial positions of the image data 116 in relation to the 3D data 112, can be performed before or after the generating 118 of the 3D model 120. However, it is performed before the embedding 126 in step d).

The 3D data or first image data may comprise pre-interventional image data. The image data 116 or second image data may comprise live images or intra-operational, or intra-interventional images.

Thus, it is possible to show current, i.e. actual, information about the situation, in combination with 3D data acquired or generated before the intervention. Thus, the 3D data can show enhanced visibility and improved perceptibility, whereas the image data 116 provides the current, i.e. live information.

As mentioned above, the 3D data may comprise X-ray CT image data, or MRI image data.

The image data 116 may be provided as 2D X-ray image data, since such image acquisition is possible with, for example, a C-arm structure with only minimally disturbing or influencing other interventional procedures.

For example, the image data 116 is provided as at least one fluoroscopic X-ray image. Preferably, at least two fluoroscopy X-ray images are acquired from different directions in order to facilitate the modeling of the device in step c).

According to an example, not further shown, following step d), a step e) is provided in which a 3D view of the reconstructed device within the 3D data is displayed to the user.

For example, as shown in FIG. 7, a 3D representation 142 of the region of interest is generated in a generation step 144 from the 3D data 112. In step d), the 3D model 120 is embedded 126 within the 3D representation in order to provide an improved model-updated 3D image 124. For example, in case the object is a patient, the 3D data 112 comprises vessel information and the data is segmented to reconstruct a tubular structure of the object for the 3D representation 142. As another example, anatomical context can be extracted from the 3D data for the 3D representation 144. For example, the reconstruction of the tubular structure comprises an aorta and iliac arteries 3D segmentation.

As will be explained also with reference to FIG. 12 et seq., the device may be deployable device, such as a stent. In the image data 116, i.e. in the second image data, the device is in the deployed state. The device may also be shown in its final state and final position.

For example, the device is an artificial heart valve in a deployed state.

According to a further exemplary embodiment, shown in FIG. 8, for step d), an expected spatial relationship 146 between the 3D model 120 and the 3D data 112 is predetermined in a predetermination step 148. For the embedding 126, the 3D model 120 is adjusted accordingly, as indicated with adjustment arrow 150.

For example, the expected relationship can comprise the location within a vessel structure, for example when placing a stent inside a vessel tree. In such case, it can be assumed that the stent itself must be placed inside a vessel structure. Thus, if the embedding would result in a location of the model of the stent such that it would only be partly placed inside a vessel structure, or even next to or outside a vessel structure, it must be assumed that this is not reflecting the actual position, but is rather based on an incorrect spatial arrangement, for example an incorrect registration step. In such case, the expected relationship can be used to adapt or modify the positioning accordingly.

It must be noted that, in the drawing, the adjustment arrow 150 enters the embedding box 126. However, according to a further example, the adjusting arrow 150 can also be provided as entering the model generation box 118 of step c), which is not further shown. It is further noted that the predetermination 148 can also be provided in combination with the transformation as explained with reference to FIG. 5. Of course, this is meant as an option only, which is why the respective arrow is shown in a dotted manner in FIG. 8.

According to a further example, shown in FIG. 9, following step d), a step e) is provided in which the model-updated 3D image 124 is displayed as display information 152 in a display step 154, wherein the model updated 3D image is displayed within the 3D representation 142 of the region of interest.

According to a further exemplary embodiment, shown in FIG. 10, predetermined features 156 of the device and/or the object or detected in the model-updated 3D image 124 in a detection step 158.

For example, the predetermined features 156 are highlighted in the model-updated 3D image 124, which is indicated, with highlighting arrow 160.

According to a further example, which can be provided alternatively or in addition to the highlighting 160 and which is shown also in FIG. 10, measurement data 162 of the predetermined features in relation to the object is determined in a determination step 164. For example, the measurement data 162 is provided to define and/or adapt a steering or guiding strategy of an intervention. The provision of the measurement data 162 is indicated with provision arrow 166, and the definition or adaption is indicated with box 162, as an example only.

For example, the device is a first part of first stent body of a stent graft and a gate of the first part is detected and the position data of the gate is used for placing a second part of a stand graft such that the two parts sufficiently overlap, which will be explained with reference to FIG. 12 et seq. The term “gate” designates an opening in the endo-prosthesis through which wiring should be achieved. The wire has to be threaded through this opening, which constitutes a complex operation due to the lack of depth perception in interventional projective images such as fluoroscopy images. This will be further explained in the description of FIGS. 12 to 15.

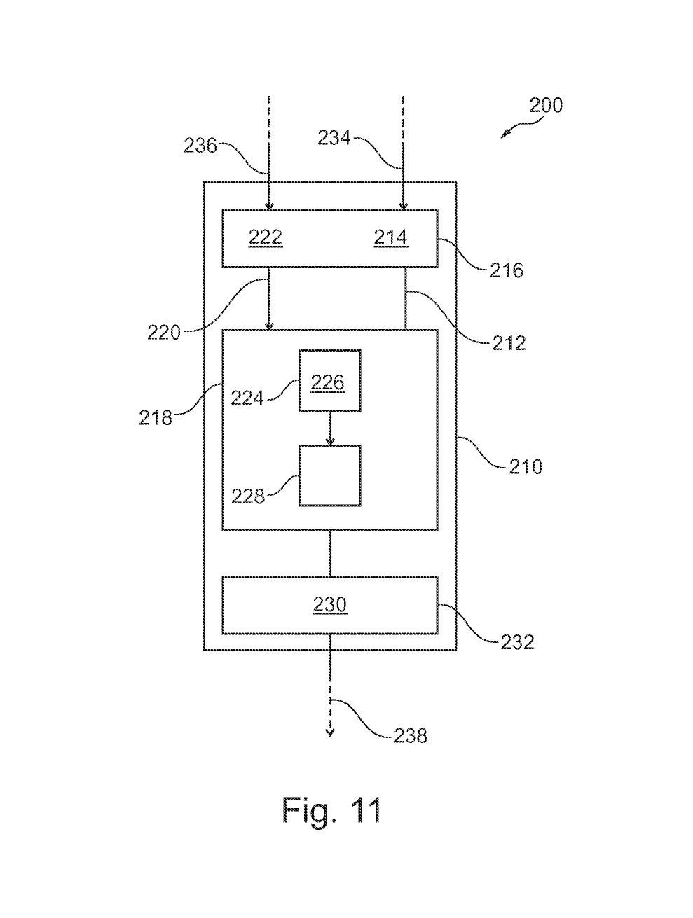

FIG. 11 shows a method 200 for operating an image processing device 210 for guidance support. The following steps are provided: In a first provision step 212, 3D data 214 of a region of interest of an object is provided from an input unit 216 to a processing unit 218. In a second provision step 220, image data 222 of at least a part of the region of interest is provided from the input unit 216 to the processing unit 218, wherein a device is arranged at least partly within the region of interest. Next, in a generating step 224, a 3D model 226 of the device is generated from the image data 222 by the processing unit 218. In an embedding step 228, the 3D model 226 is embedded within the 3D data 214 by the processing unit 218 to provide a model-updated 3D image 230 via an output unit 232.

The 3D data 214 may be provided from an external data source, such as a storage medium, as indicated with a first provision arrow in a dotted manner, with reference numeral 234. The image data 222 may be provided, for example, from an image acquisition device, as indicated with a second dotted provision arrow 236. The model-updated 3D image 230 may be provided, for example, to display device, as indicated with dotted output arrow 238.

An example for an application of the above-mentioned procedures will be described in the following with reference to FIGS. 12 to 15.

In endovascular surgery procedures, the so-called endovascular aneurism repair (EVAR) is an important interventional procedure. FIG. 12 shows a vessel structure 300 with an aneurism 310. As also indicated in FIG. 12, a stent graft 312 is shown, which, for example, has been inserted in the aorta through a small incision in the femoral artery. It is then deployed in the abdominal aortic aneurism, for example just below the renal arteries, indicated with reference numeral 314, and covers the aortic bifurcation, indicated with reference numeral 316. The stent graft 312 is therefore composed of two parts. A main body 313, as shown in FIG. 12, covering the aorta and one iliac artery is first positioned. It has a gate 318, i.e. an entry opening, in which a second part 324, shown in FIG. 14, is then inserted. To this aim, the interventionist has to thread a guide wire 320 (see FIG. 13) into the gate under fluoroscopy guidance according to known procedures.

In order to facilitate the insertion of the guide wire 320 into the gate 318 of the stent main body 313, the deployed prosthesis, as shown in FIGS. 12 and 13, is modeled in 3D from one or several fluoroscopy images, as described above. The modeling result is then embedded within a preoperative CT scan, for example. In this way, the deployed device can be viewed in 3D within its anatomical context; in particular, the relative position of the gate 318 and of the aortic wall, indicated with reference numeral 322, can be properly displayed, which indicates the appropriate steering of the wire 320.

According to one example, to this end, only the gate needs to be modeled and embedded in the pre-operative CT data. The gate appears in the fluoroscopy images as an ellipse-shape wiry structure. As such it can be automatically detected (for instance relying on a gradient-based Hough-transform for the finding of a parametric shape such as an ellipse), and it can be segmented in two images corresponding to distinct angulations. From these two segmentation results (two elliptical 2D lines), a 3D elliptical line can be computed, the projections of which onto the two originating image planes correspond to the observed gates in those images.

The CT data can be processed such that mainly the vessel boundaries are represented (for instance as a surface or as a mesh). The embedding then consists in representing the 3D elliptical line modeling the device (here the gate) together with the vessel boundaries.

Of course this joint representation should be achieved in a common frame of reference for both the model and the pre-operative data. This might require co-registration of the model with the pre-operative data in case the frames of reference of these two data sources do not natively correspond to each other. In particular this is the case when combining CT and X-Ray-originated data. This is not the case when the 3D data are created with a C-arm CT technique (rotational X-Ray). In this case the 3D data and the model, which is computed from 2D X-ray projections, originate from the same system and can be natively expressed in the same frame of reference, making co-registration superfluous

In addition to the gate, the guide-wire itself (or simply its distal tip) can also be modeled as a 3D line. One can then visualise within a single representation the triplet vessel-wall, prosthesis entry point, intervention device to be threaded through this entry point and potentially taking support onto the vessel walls.

Thus, additional image acquisition steps under X-ray fluoroscopy are not necessary in case the wire tip and its relative depth with respect to the gate's location cannot be properly estimated in the projective view of a fluoroscopy image. Rather, this information, which is of crucial importance to the surgeon, since threading the prosthesis gate is one of the most delicate phases of the intervention, is provided with the respective information by the model-updated 3D image, generated and embedded according to the invention. In other words, the insertion of a guide wire 320, as shown in FIG. 13, is facilitated with the above described invention, such that, as can be seen in FIG. 14, the second part of the stent 324, i.e. also called the contralateral stent, can be inserted and deployed such that it has a sufficient overlap with the stent body 313.

According to the invention, it is also possible to facilitate the steering of a respective short extension piece 326, as third part, shown in FIG. 15 in its deployed state with a sufficient overlap with the main stent body 313. The second part 324 is also referred to as long contralateral extension piece.

It must be noted that, according to an exemplary embodiment of the invention, the model-updated 3D representation is only valid as long as the modeled objects correspond with the live 2D projections. The gate being rather static, this remains true for a long period. Gate-upgraded 3D data can then be computed only once and can be used for the gate passing intervention step. But the guide-wire is naturally steered and does not remain static. This implies that joint gate-plus-wire modeling is only valid when corresponding live images are available. In particular this is the case with a bi-plane system that can constantly produce pairs of projections that can be used for the constant generation of gate-plus-wire modeling and 3D upgrading.

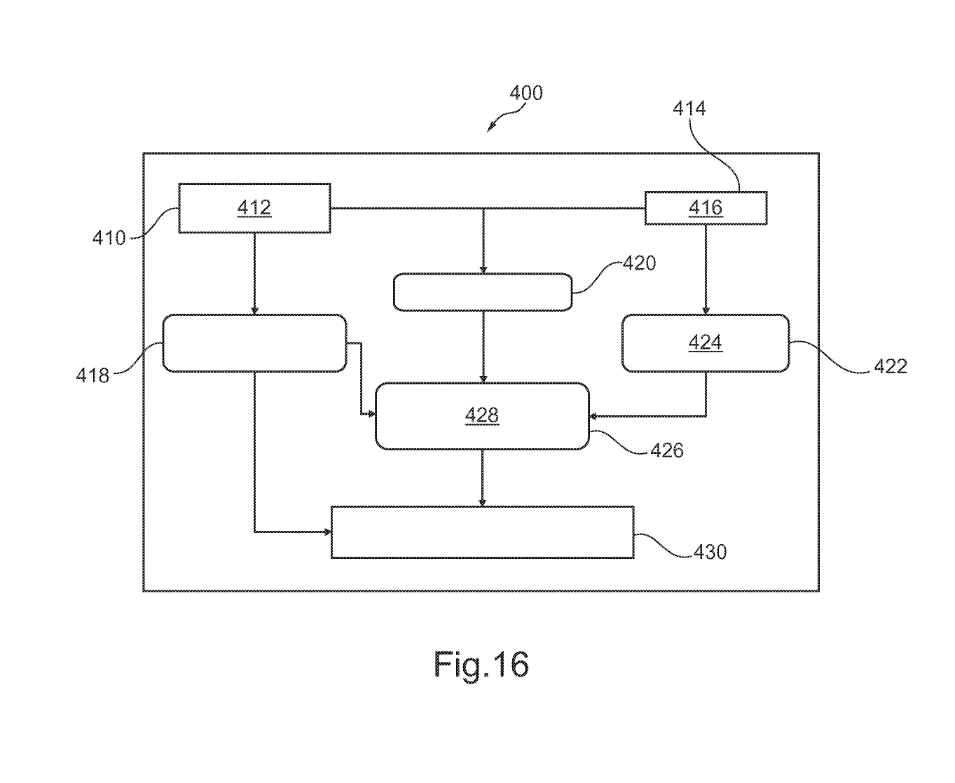

FIG. 16 shows a further exemplary embodiment of a method 400 according to the invention, with reference to the above described endovascular aneurism repair, which background is shown in FIGS. 12 to 15. As a first input 410, a pre-interventional CT scan 412 of the region of interest where the stent will be deployed, is provided. For example, the aorta is contrasted. Further, depending on a 2D/3D registration method, the scan region may also have to include other regions as the spine or the pelvis. 3D ridging modalities fulfilling these pre-requisites such as MRI could also be used.

As a second input 414, live images 416 from an X-ray system are provided, for example fluoroscopic images taken from a reduced number of views after the deployment of the first part of the stent graft. These are the usual views used to assess the current situation. From the pre-interventional CT-scan 412, a segmentation 418 is provided, segmenting aorta and iliac arteries in 3D. This can be achieved by automatic or semi-automatic algorithmic solutions extracting tubular structures in a 3D data 112 volume. Further, segmentation of abdominal aortic aneurism can also be applied. The pre-interventional CT scan 412 and the live images 416 are further registered in a 2D/3D registration step 420. Therewith, the position of the pre-interventional CT scan, or of the 3D aorta segmentation, in the X-ray system frame of reference is found. 2D/3D registration algorithms are used to retrieve that particular position from one or several X-ray projections. For example, the vertebrae and the pelvis could be used to register the whole CT scan. Angiograms from the aorta could also be used to register the 3D segmented aorta.

According to the invention, in a modeling step 422, the stent graft main body is modeled in 3D. It is noted that the shape of a stent graft, principally at the gate level, is simple and quite regular, i.e. a tubular structure with a bifurcation. It is possible to use assumptions about its shape, such that it can be modeled from reduced set of fluoroscopic images. The result is a 3D model 424 obtained in the X-ray system frame of reference. The 3D segmentation and the 3D model are then provided to an adjustment step 426 in which the stent model is adjusted within the 3D reconstruction of the aorta. Depending on the 2D/3D registration algorithm, the model stent could not be properly positioned within the 3D segmentation of the aorta. Therefore, a residual transformation, for example, to place the stent within the 3D segmentation of the aorta, is computed. As a result, an adjusted model within the 3D reconstruction is provided, also referred to with reference numeral 428.

Further, the 3D segmentation is used in an embedding step 430, in which the 3D view of the stent graft is embedded within the 3D segmentation of the aorta. The interventionist can then use this particular view to assess the position of the gate within the aorta and adapt its strategy to insert the guide wire.

According to a further example, also with reference to FIG. 16, the intervention wire tip can also be part of the 3D model, and once embedded in the 3D data, the relative positions of the stent (in particular of the gate), of the tool (in particular of the wire tip), and of the anatomy (in particular of the vessel borders) is made clear, and remain valid as long as the intervention tool has not been steered. But since the full process (modeling+adjustment) can be repeated on incoming live data 416, in particular originating from a bi-plane system, the upgraded 3D view can be constantly refreshed and can remain relevant.

It is noted that according to a further example, the method as shown in FIG. 16 is provided without the segmentation step 418 and without the adjustment step 426. Instead, the 2D/3D registration 420 is provided directly to the embedding step 430, as is also the case for the 3D modeling 422, which is also provided directly to the embedding step 430 instead.

According to a further exemplary embodiment of the invention, although not shown, the modeling of a device from live data into preoperative CT is also applied in other interventions, such as transcatheter valve implantation.

In another exemplary embodiment of the present invention, a computer program or a computer program element is provided that is characterized by being adapted to execute the method steps of the method according to one of the preceding embodiments, on an appropriate system.

The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the Internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An image processing device (10) for guidance support, comprising:
a processing unit (12);
an input unit (14); and
an output unit (16);
wherein the input unit is adapted to provide 3D data (18) of a region of interest of an object; and to provide image data (20) of at least a part of the region of interest, wherein a device is arranged at least partly within the region of interest; wherein the device is a stent;
wherein the processing unit comprises a generation unit (26) to generate a 3D model (28) of the device from the image data, wherein shape assumptions of the stent are provided to facilitate the modeling;
wherein the processing unit comprises an embedding unit (30) to embed the 3D model within the 3D data; and
wherein the output unit is adapted to provide a model-updated 3D image (32) with the embedded 3D model.

2. Device according to claim 1, wherein the image data comprises at least one 2D image and wherein the generation unit is adapted to generate the 3D model from the at least one 2D image;
and wherein the generation unit is adapted to generate a 3D representation (36) of the region of interest from the 3D data; and
wherein the embedding unit is adapted to embed the 3D model within the 3D representation.

3. A medical imaging system (50) for providing guidance support, comprising:
an image acquisition arrangement (52);
a device (10) according to claim 1; and
a display unit (54);
wherein the image acquisition arrangement is adapted to acquire the image data and to provide the data to the processing unit;
wherein the output unit is adapted to provide the model-updated 3D image to the display unit; and
wherein the display unit is adapted to display the model-updated 3D image.

4. System according to claim 3, wherein the image acquisition arrangement is an X-ray imaging arrangement (56) with an X-ray source (58) and an X-ray detector (60); and
wherein the X-ray imaging arrangement is adapted to provide 2D X-ray images as image data.

5. A method (100) for guidance support, comprising the following steps:
a) providing (110) 3D data (112) of a region of interest of an object;
b) providing (114) image data (116) of at least a part of the region of interest, wherein a device is located at least partly within the region of interest; wherein the device is a stent;
c) generating (118) a 3D model (120) of the device from the image data; wherein shape assumptions of the stent are provided to facilitate the modeling;
d) providing (122) data for a model-updated 3D image (124) by embedding (126) the 3D model within the 3D data.

6. Method according to claim 5, wherein the 3D data in step a) comprises a first frame of reference (128) and the image data in step b) comprises a second frame of reference (130);
wherein for the embedding in step d), a transformation (132) between the first frame of reference and the second frame of reference is determined (134); and
wherein the transformation is applied (136) to the 3D model.

7. Method according to claim 5, wherein the image data (116) comprises at least one 2D image.

8. Method according to claim 5, wherein a 3D representation (142) of the region of interest is generated (144) from the 3D data; and wherein in step d), the 3D model is embedded within the 3D representation.

9. Method according to claim 5, wherein for step d), an expected spatial relationship (146) between the 3D model and the 3D data is predetermined (148); and wherein for the embedding, the 3D model is adjusted (150) accordingly.

10. Method according to claim 8, wherein, following step d), a step e) is provided in which the model-updated 3D image is displayed (154) to a user within the 3D representation of the region of interest.

11. Method according to claim 5, wherein predetermined features (156) of the device and/or the object are detected (158) in the model-updated 3D image; and wherein the predetermined features are highlighted (160) in the model-updated 3D image.

12. Method according to claim 5, wherein predetermined features (165) of the device and/or the object are detected (158) in the model-updated 3D image; and
wherein measurement data (162) of the features in relation to the object is determined (164); and
wherein the measurement data is provided (166) to define and/or adapt (168) a steering or guiding strategy of an intervention.

13. A method (200) for operating an image processing device (210) for guidance support, comprising the following steps:
providing (212) 3D data (214) of a region of interest of an object from an input unit (216) to a processing unit (218);
providing (220) image data (222) of at least a part of the region of interest from the input unit to the processing unit, wherein a device is arranged at least partly within the region of interest, wherein the device is a stent;
generating (224) a 3D model (226) of the device from the image data by the processing unit; wherein shape assumptions of the stent are provided to facilitate the modeling;
embedding (228) the 3D model within the 3D data by the processing unit to provide a model-updated 3D image (230) via an output unit (232).

14. Computer program element for controlling an apparatus according to claim 1, which, when being executed by a processing unit.

15. Computer readable medium having stored the program element of claim 14.
